(19) United States
(12) Patent Application Publication
(10) Pub. No.: US 2025/0339077 A1
(43) Pub. Date: Nov. 6, 2025

(54) ELECTRO-ANATOMICAL MAPPING AND ANNOTATION PRESENTED IN ELECTROPHYSIOLOGICAL PROCEDURES

(71) Applicant: Biosense Webster (Israel) Ltd., Yokneam (IL)

(72) Inventors: Assaf GOVARI, Haifa (IL); Andres Claudio ALTMANN, Haifa (IL); Vadim GLINER, Haifa (IL)

(73) Assignee: Biosense Webster (Israel) Ltd., Yokneam (IL)

(21) Appl. No.: 19/273,466

(22) Filed: Jul. 18, 2025

Related U.S. Application Data
(63) Continuation of application No. 17/400,402, filed on Aug. 12, 2021, now Pat. No. 12,364,426.

(51) Int. Cl. A61B 5/287; A61B 5/367

(52) U.S. Cl. CPC A61B 5/287; A61B 5/367

(57) ABSTRACT

A catheter includes: (a) a shaft for insertion into a heart of a patient, (b) an expandable distal-end assembly, which is coupled to the shaft and is configured to make contact with tissue of the heart, (c) at least first and second electrocardiogram (ECG) electrodes, which are coupled to an outer surface of the expandable distal-end assembly, and when placed in contact with the tissue, are configured to sense ECG signals in the tissue, and (d) a reference electrode, which is positioned within an inner volume of the distal-end assembly, and in an expanded position of the distal-end assembly, the reference electrode: (i) has no physical contact with the tissue, and (ii) is positioned at a first distance from the first ECG electrode and at a second distance from the second ECG electrode, and the difference between the first and second distances is smaller than a predefined threshold.

### RELATED APPLICATION INFORMATION

The present application is a continuation of prior filed U.S. patent application Ser. No. 17/400,402 filed on Aug. 12, 2021 (Attorney Docket No. 253757.000443 (BIO6555USNP1)), of which is hereby incorporated by reference as if set forth in full herein.

### FIELD OF THE INVENTION

The present invention relates generally to medical devices, and particularly to methods and systems for improving sensing of unipolar signals in patient heart, and improving accuracy of annotations presented to a user.

### BACKGROUND OF THE INVENTION

Various techniques for analyzing and annotating electrophysiological signals have been published.

For example, U.S. Patent Application Publication 2020/0367751 describes a device for detecting points and/or regions of rotational electrophysiological activity in or on a heart. The device comprises an input for receiving spatiotemporal electrophysiological data corresponding to a plurality of spatial locations in or on the heart, a time feature extractor for providing time values indicative of times of occurrence of a predetermined feature of a plurality of electric potential waveforms at the spatial location, a mapping unit for providing pairs of adjacent spatial locations, a directed graph generator for generating a directed graph comprising directed edges, and a topological feature analyzer.

U.S. Patent Application Publication 2018/0303414 describes systems, devices and methods for performing precise treatment, mapping, and/or testing of tissues.

### SUMMARY OF THE INVENTION

An embodiment of the present invention that is described herein provides a catheter, including (a) a shaft for insertion into a heart of a patient, (b) an expandable distal-end assembly, which is coupled to the shaft and is configured to make contact with tissue of the heart, (c) at least first and second electrocardiogram (ECG) electrodes, which are coupled to an outer surface of the expandable distal-end assembly, and when placed in contact with the tissue, are configured to sense ECG signals in the tissue, and (d) a reference electrode, which is positioned within an inner volume of the distal-end assembly, and in an expanded position of the distal-end assembly, the reference electrode: (i) has no physical contact with the tissue, and (ii) is positioned at a first distance from the first ECG electrode and at a second distance from the second ECG electrode, and a difference between the first and second distances is smaller than a predefined threshold.

In some embodiments, the expandable distal-end assembly includes at least first and second splines, and the first and second ECG electrodes are coupled to the first and second splines, respectively. In other embodiments, in the expanded position, the reference electrode is positioned at least 9 mm from the tissue. In yet other embodiments, in the expanded position, the predefined threshold is smaller than 1 mm.

In an embodiment, at least one of the ECG signals includes a unipolar ECG signal, which is sensed in the tissue relative to the reference electrode. In another embodiment, the catheter includes a processor, which is configured to calculate, based on the ECG signals sensed at one or more positions on the tissue, one or more respective local activation times (LATs), which are indicative of an electrophysiological wave propagating in the tissue. In yet another embodiment, based on the calculated LATs, the processor is configured to display to a user at least an annotation indicative of a timing error in a rhythm of the heart.

In some embodiments, the processor is configured to display at least the annotation over a graph of at least one of the ECG signals. In other embodiments, the processor is configured to display at least the annotation in a bar graph. In yet other embodiments, the processor is configured to display in the bar graph at least a first annotation indicative of a first type of the timing error, and a second different annotation, which is indicative of a second type of the timing error, different from the first type.

There is additionally provided, in accordance with an embodiment of the present invention, a method including inserting, into a heart of a patient, an expandable distal-end assembly having an expanded position for making contact with tissue of the heart, the distal-end assembly includes: (i) at least first and second electrocardiogram (ECG) electrodes, which are coupled to an outer surface of the distal-end assembly for sensing ECG signals with the tissue when placed in contact with the tissue, and (ii) a reference electrode, which is positioned within an inner volume of the distal-end assembly, and in the expanded position: (a) has no physical contact with the tissue, and (b) is positioned at a first distance from the first ECG electrode and at a second distance from the second ECG electrode, and a difference between the first and second distances is smaller than a predefined threshold. The distal-end assembly is expanded for placing at least the first and second ECG electrodes in contact with the tissue, and ECG signals are sensed in the tissue using at least the first and second ECG electrodes.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a catheter-based position-tracking and ablation system, in accordance with an embodiment of the present invention;

FIG. 2 is a schematic, side view of a distal-end assembly of a catheter, in accordance with an embodiment of the present invention; and

FIG. 3 is a flow chart that schematically illustrates a method for sensing unipolar ECG signals in tissue, in accordance with an embodiment of the present invention.

### DETAILED DESCRIPTION OF EMBODIMENTS

### Overview

Some electrophysiological (EP) procedures require electro-anatomical (EA) mapping of heart tissue using electrodes for acquiring unipolar electrocardiogram (ECG) signals in the tissue in question. The term “unipolar signal” refers to a signal acquired by a sensing electrode, which is placed in contact with the tissue in question, relative to a reference electrode.

In principle, it is possible to measure the ECG signals relative to a reference annotation measured on one or more external reference electrodes positioned on the patient skin using a suitable patch. Moreover, it is possible to calculate, based on the acquired ECG signals, values local activation times (LAT) of an EP wave propagating in the tissue in question. For example, a unipolar signal sensed by a given ECG electrode may constitute the difference between the potential at the given electrode 88 and the Wilson Central Terminal (WCT) set of body surface electrodes positioned on the right arm, left arm, and left leg of the patient. However, using a set of one or more external reference electrode may introduce noise, inaccuracy and/or instability to the sensing of the ECG signals. These inaccuracy and/or instability may result in wrong calculation of the LAT values, and therefore wrong binning and wrong annotations of arrhythmias based on the ECG signals and calculated LAT values.

In some embodiments, a system for improving the sensing of ECG signals comprises a catheter and a processor. The catheter comprises a shaft for insertion into a heart of a patient, an expandable distal-end assembly coupled to the shaft, multiple ECG electrodes, and one or more reference electrodes. In some embodiments, the distal-end assembly is configured to have a collapsed position for being moved to a target location, and an expanded position for making contact with the tissue in question. In the present example the catheter comprises at least first and second ECG electrodes, which are coupled to an outer surface of the expandable distal-end assembly, and, when placed in contact with the tissue, are configured to sense ECG signals in the tissue.

In some embodiments, the catheter comprises a single reference electrode, which is positioned within the inner volume of the distal-end assembly, for example, on a shaft positioned at the center of the distal-end assembly when expanded to the expanded position. Moreover, in the expanded position of the distal-end assembly, the reference electrode has no physical contact with the tissue and in the present example, has a distance of at least 9 mm from the tissue in question. Moreover, the reference electrode is positioned at a first distance from the first ECG electrode and at a second distance from the second ECG electrode. The first and second distances are typically similar, in other words, the difference between the first and second distances is smaller than a predefined threshold, e.g., about 1 mm.

In some embodiments, the processor is configured to receive from the ECG electrodes in contact with the tissue, unipolar ECG signals measured relative to the reference electrode, and to calculate one or more local activation times (LATs), which are indicative of the EP wave propagating in the tissue.

In some embodiments, based on the calculated LATs, the processor is configured to display to a user of the system one or more annotations indicative of respective one or more timing errors in the rhythm of the heart. The processor is further configured to display the one or more annotations over one or more graphs of the unipolar ECG signals. Additionally or alternatively, the processor is configured to display a bar graph having two or more types of timing errors resulting in wrong annotations.

The disclosed techniques improve the quality of unipolar ECG signals sensed in the patient heart, and the accuracy of annotations presented to a user during an electrophysiological procedure. Moreover, the disclosed techniques improve the quality of EA mapping and the success rate of ablation procedures carried out based on the EA mapping.

### System Description

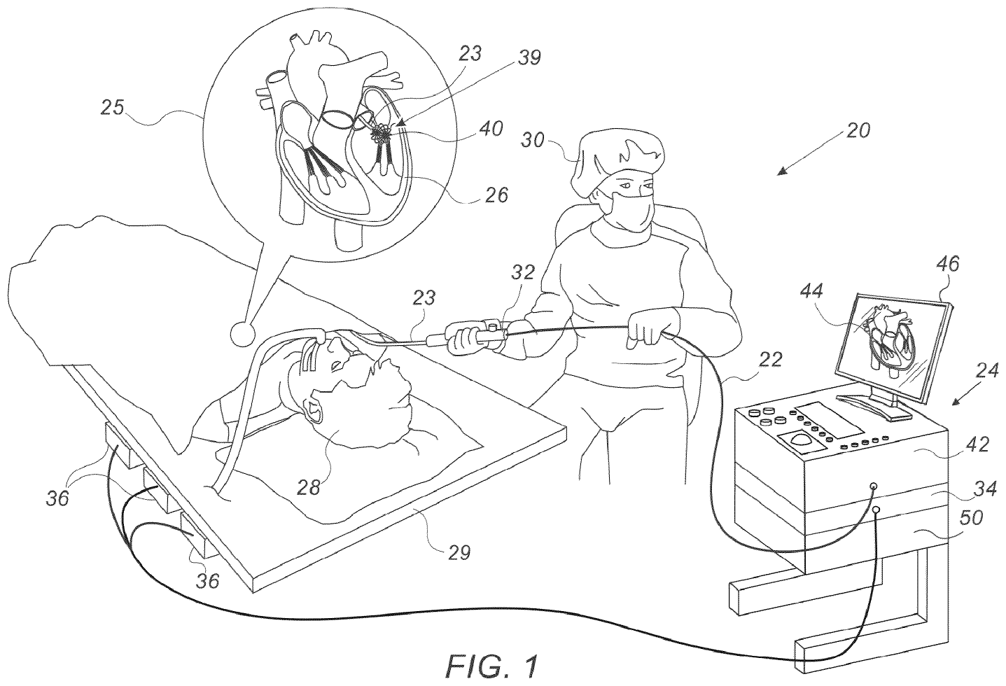

FIG. 1 is a schematic, pictorial illustration of a catheter-based position-tracking and ablation system 20, in accordance with an embodiment of the present invention. In some embodiments, system 20 comprises a catheter 22, in the present example an expandable cardiac catheter having a basket shape, and a control console 24. In the embodiment described herein, catheter 22 may be used for any suitable therapeutic and/or diagnostic purposes, such as but not limited to electro-anatomical (EA) mapping and/or ablation of tissue in a heart 26.

In some embodiments, console 24 comprises a processor 42, typically a general-purpose computer, with suitable front end and interface circuits for receiving signals from catheter 22 and for controlling other components of system 20 described herein. Processor 42 may be programmed in software to carry out the functions that are used by the system, and is configured to store data for the software in a memory 50. The software may be downloaded to console 24 in electronic form, over a network, for example, or it may be provided on non-transitory tangible media, such as optical, magnetic or electronic memory media. Alternatively, some or all of the functions of processor 42 may be carried out using an application-specific integrated circuit (ASIC) or any suitable type of programmable digital hardware components.

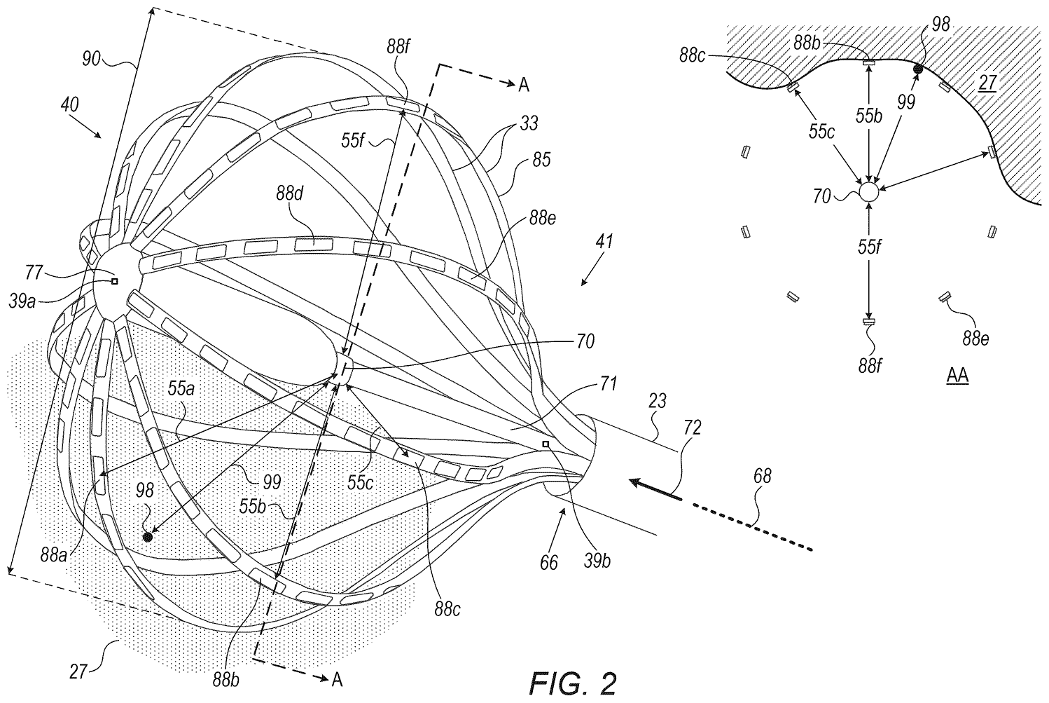

Reference is now made to an inset 25. In some embodiments, catheter 22 comprises a distal-end assembly 40 having multiple splines (shown in detail in FIG. 2 below), and a shaft 23 for inserting distal-end assembly 40 to a target location for ablating tissue in heart 26. During an ablation procedure, physician 30 inserts catheter 22 through the vasculature system of a patient 28 lying on a table 29. Physician 30 moves distal-end assembly 40 to the target location in heart 26 using a manipulator 32 near a proximal end of catheter 22, which is connected to interface circuitry of processor 42.

In some embodiments, catheter 22 comprises one or more position sensor(s) 39 of a position tracking system, which is coupled to the distal end of catheter 22, e.g., in close proximity to distal-end assembly 40. In the present example, position sensors 39 comprise a magnetic position sensor, but in other embodiments, any other suitable type of position sensor (e.g., other than magnetic-based) and corresponding position tracking system may be used.

Reference is now made back to the general view of FIG. 1. In some embodiments, during the navigation of distal-end assembly 40 in heart 26, processor 42 receives signals from magnetic position sensors 39 in response to magnetic fields from external field generators 36, for example, for the purpose of measuring the position of distal-end assembly 40 in heart 26. In some cases distal-end assembly 40 comprises two position sensors, so as to control the level of expansion of distal-end assembly 40. In some embodiments, console 24 comprises a driver circuit 34, which is configured to drive magnetic field generators 36. Magnetic field generators 36 are placed at known positions external to patient 28, e.g., below table 29.

In some embodiments, processor 42 is configured to display, e.g., on a display 46 of console 24, the tracked position of distal-end assembly 40 overlaid on an image 44 of heart 26.

The method of position sensing using external magnetic fields is implemented in various medical applications, for example, in the CARTO™ system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publications 2002/0065455 A1, 2003/0120150 A1 and 2004/0068178 A1, whose disclosures are all incorporated herein by reference.

### Distal-End Assembly Having a Central Reference Electrode

FIG. 2 is a schematic, side view of distal-end assembly 40, in accordance with an embodiment of the present invention.

In some embodiments, distal-end assembly 40 is coupled to shaft 23 and is navigated by physician 30 to be placed in contact with tissue in question of heart 26 or with tissue of any other organ of the patient, as described in FIG. 1 above.

In some embodiments, distal-end assembly 40 is expandable, in the present example has a basket shape, but in other embodiments, distal-end assembly 40 may have any other suitable type of an expandable assembly.

In some embodiments, distal-end assembly 40 has splines 33 coupled between a proximal apex 66 and a distal apex 77 of distal-end assembly 40. In the present example, at least one of and typically all splines 33 are made from any suitable biocompatible material, and are electrically isolated from one another, e.g., by apexes 66 and 77 or using any other suitable electrically insulating apparatus.

In some embodiments, at least one of and typically all splines 33 have electrocardiogram (ECG) electrodes, referred to herein as electrodes 88 for brevity. In the present example, each spline 33 has one or more electrodes 88 coupled to an outer surface 85 of the spline. In the context of the present disclosure and in the claims, the term “outer surface” refers to a surface of splines 33 intended to be placed in contact with tissue 27 of heart 26. Electrodes 88 are configured to sense ECG signals in tissue 27. Note that at least one of electrodes 88 is configured to sense electro gram (EGM) signals in tissue 27 of heart 26.

In some embodiments, apexes 66 and 77 are movable relative to one another so as to expand and collapse distal-end assembly 40. For example, apex 66 is moved distally in a direction 72, which is parallel to an axis 68 of catheter 22, so as to expand distal-end assembly 40. Similarly, apex 66 is moved proximally along axis 68 (i.e., opposite to direction 72), so as to collapse distal-end assembly 40. In the present example, axis 68 constitutes a longitudinal axis of both catheter 22 and distal-end assembly 40.

In some embodiments, distal-end assembly 40 may have two position sensors 39a and 39b, which are coupled to, or in adjacent to, apexes 77 and 66, respectively. Based on the position signals received from position sensors 39a and 39b, processor 42 calculates the distance between apexes 66 and 77, and thereby, the expansion-level of distal-end assembly 40.

In some embodiments, distal-end assembly 40 has a reference electrode 70, which is positioned within an inner volume 41 of distal-end assembly 40. In the context of the present disclosure and in the claims, the term “inner volume” refers to the space confined between splines 33. Thus, the larger the expansion-level of distal-end assembly 40, the larger the space confined within inner volume 41.

In some embodiments, reference electrode 70 has no physical contact with tissue 27. In the example of FIG. 2, reference electrode 70 is coupled to a shaft 71, which is extended along axis 68, so that reference electrode 70 is positioned at the center of inner volume 41, and therefore, is also referred to herein as a central electrode.

In some embodiments, when distal-end assembly 40 is placed in contact with tissue 27 of heart 26, one or more splines 33 and electrodes 88 are placed in contact with tissue 27. In such embodiments, reference electrode 70 is positioned at a distance 99 from a point 98, which represents a point on the surface of tissue 27 that is in the closest proximity (from among all the other points on the surface of tissue 27) to reference electrode 70.

In some embodiments, reference electrode 70 is positioned at distances 55a, 55b, 55c and 55f, from electrodes 88a, 88b, 88c and 88f, respectively. In the context of the present disclosure, the term electrode(s) 88 refers to any electrode from among electrodes 88a, 88b, 88c, 88d, 88e, and 88f. When distal-end assembly 40 is fully expanded and has a maximal diameter 90, all distances 55 (e.g., distances 55a, 55b, 55c and 55f) have approximately the same size. For example, the difference between any pair of distances selected from among distances 55a, 55b, 55c and 55f, is smaller than a predefined threshold (e.g., about 1 mm), whereas at the fully expanded position, diameter 90 may have a size between about 18 mm and 30 mm, or any other suitable size. Note that the difference between any pair of distances may also be defined in percentage of maximal diameter 90 when distal-end assembly 40 is in the fully expanded position.

In the context of the present disclosure and in the claims, the terms “about” or “approximately” for any numerical values or ranges indicate a suitable dimensional tolerance that allows the part or collection of components to function for its intended purpose as described herein. Moreover, the terms “about” or “approximately” may also be used for comparing between a physical dimension, or a measurable feature of two or more elements, and the terms “about” or “approximately” may indicate a suitable dimensional tolerance between the compared elements.

Reference is now made to a sectional-view AA of distal-end assembly 40 in the fully expanded position shown in the general view of FIG. 2.

In some embodiments, electrodes 88b and 88c are placed in contact with tissue 27, electrode 55f is not in contact with tissue 27, and distances 55b, 55c and 55f have a similar size. Note that electrode 88a is also placed in contact with tissue 27, but is not in the plane of sectional-view AA, and therefore, is not shown therein. Moreover, distance 99 represents the minimal distance between reference electrode 70 and tissue 27. In the present example, distance 99 is larger than about 9 mm.

In some embodiments, distal-end assembly 40 is designed such that in an expanded position, reference electrode 70 is positioned at a similar distance from all electrodes 88 (e.g., electrodes 88a, 88b, 88c, 88d, 88e, and 88f). The term “similar distance” refers to a difference between distances 55, which is smaller than a predefined threshold, such as the threshold defined above, or any other suitable threshold. More specifically, in the plane shown in sectional-view AA, all the distances between central electrode 70 and electrodes 88 have a similar size (e.g., the difference between the sizes are smaller than the aforementioned threshold). Moreover, central electrode 70 is also located at a predefined minimal distance from tissue 27. In the present example, at a distance larger than about 9 mm, which is represented in sectional-view AA by distance 99.

Reference is now made back to the general view of FIG. 2. In some embodiments, during electro-anatomical mapping of tissue 27 in heart 26, ECG electrodes 88 are configured to sense ECG signals in tissue 27, and processor 42 is configured to estimate, based on the sensed ECG signals, the local activation time (LAT) values of EP waves propagating in heart 26 in close proximity to and also along tissue 26. Processor 42 calculates the LAT values, inter alia, based on the timing of the multiple ECG signals acquired by and received from multiple ECG electrodes 88.

In principle, it is possible to measure the ECG signals and to calculate the LAT values relative to a reference annotation measured on one or more external reference electrodes (not shown) positioned on the skin of patient 28 using a suitable patch. For example, a unipolar signal sensed by a given ECG electrode 88 may constitute the difference between the potential at given electrode 88 and the Wilson Central Terminal (WCT) set of body surface electrodes (not shown) positioned on the right arm, left arm, and left leg of patient 28. However, using a set of one or more external reference electrode may introduce noise, inaccuracy and/or instability to the sensing of the ECG signals. These inaccuracy and/or instability may result in wrong calculation of the LAT values, and therefore wrong binning and wrong annotations of the ECG signals and calculated LAT values.

In some embodiments, the accuracy and stability of the calculated LAT and of the binning and annotation of the ECG signals are improved by sensing the unipolar signals between reference electrode 70 and electrodes 88 placed in contact with tissue 27.

In other embodiments, in addition to or instead of sensing unipolar ECG signals between reference electrode 70 and electrodes 88 placed in contact with tissue 27, reference electrode 70 may be used, for example, as a reference electrode for applying, e.g., to tissue 27, a unipolar ablation signal between one or more ablation electrodes (not shown) of a given spline 33 and reference electrode 70. Note that in such embodiments (i.e., ECG sensing and tissue ablation), reference electrode 70 is not intended to be placed in contact with tissue 27 and the one or more electrodes of given spline 33 are placed in contact with tissue 27.

These particular configurations of system 20 and distal-end assembly 40 are shown by way of example, in order to illustrate certain problems that are addressed by embodiments of the present invention and to demonstrate the application of these embodiments in enhancing the performance of such a system and electrophysiological applications. Embodiments of the present invention, however, are by no means limited to this specific sort of example system or example applications, and the principles described herein may similarly be applied to other sorts of medical system, using any other suitable configuration of distal-end assembly having any suitable arrangement of sensing and/or ablation electrodes, and a reference electrodes that, at least during the procedure, has no physical contact with the tissue in question.

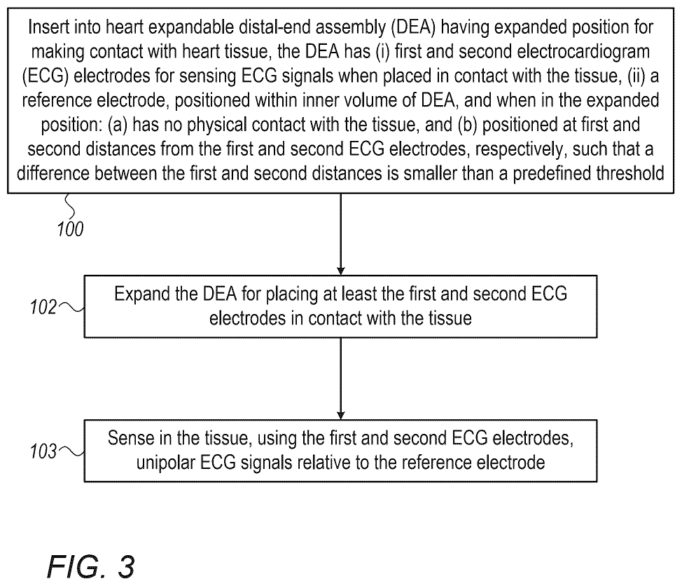

FIG. 3 is a flow chart that schematically illustrates a method for sensing unipolar ECG signals in tissue 27, in accordance with an embodiment of the present invention.

The method begins at a catheter insertion step 100, with physician 30 inserting distal-end assembly 40 into a cavity of heart 26. Distal-end assembly 40 has: (i) a collapsed position for moving catheter 22 to a target location in tissue 27 of heart 26, and (ii) an expanded position for placing electrodes 88 in contact with tissue 27, or with any other tissue in question. In the example of FIG. 2, at least electrodes 88a, 88b and 88c are placed in contact with tissue 27.

In some embodiments, distal-end assembly 40 comprises reference electrode 70, which is positioned within inner volume 41 of distal-end assembly 40, and in the expanded position of distal-end assembly 40, reference electrode 70 has no physical contact with tissue 27, and typically distance 99 is larger than about 9 mm.

In some embodiments, reference electrode 70 is positioned at distances 55a, 55b, 55c and 55f from electrodes 88a, 88b, 88c and 88f, respectively. The difference between any pair of distances 55a, 55b, 55c and 55f is smaller than a predefined threshold, e.g., about 1 mm. Moreover, when distal-end assembly 40 is expanded (e.g., as shown in FIG. 2 above), reference electrode 70 has no physical contact with tissue 27, and in the present example, reference electrode 70 is positioned at a distance of at least 9 mm from tissue 27.

At an electrode placement step 102, after positioning distal-end assembly 40 in close proximity to tissue 27, physician 30 uses manipulator 32 or any other suitable control knob for expanding distal-end assembly 40 to an expanded position, so that at least electrodes 88a, 88b and 88c are placed in contact with tissue 27, as depicted, for example, in FIG. 2 above.

At a unipolar signal sensing step 103 that concludes the method, processor 42 receives from at least electrodes 88a, 88b and 88c, unipolar ECG signals measured relative to reference electrode 70.

In some embodiments, based on the sensed unipolar ECG signals, processor 42 is configured to calculate one or more local activation times (LATs), which are indicative of the EP wave propagating in tissue 27. Based on the calculated LATs, processor 42 is further configured to display to physician 30 one or more annotations indicative of respective one or more timing errors identified in the rhythm of heart 26. Moreover, processor 42 is configured to display the one or more annotations over one or more graphs of the unipolar ECG signals.

Although the embodiments described herein mainly address electro-anatomical mapping and analysis in electrophysiological applications, the methods and systems described herein can also be used in other applications.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

What is claimed is:

1. A system, comprising:
one or more processors; and
a memory storing instructions that, when executed by the one or more processors are configured to cause the system to:
receive electrophysiological signals from a first electrode and a second electrode positioned on an expandable distal-end assembly;
calculate, based on the electrophysiological signals, one or more respective local activation times (LATs) which are indicative of an electrophysiological wave propagating in tissue;
identify, based at least in part on the calculated one or more respective LATs, a timing error of a rhythm of a heart of a patient; and
output for display an annotation over a graph of at least one electrocardiogram (ECG) signal, the annotation being indicative of the timing error identified in the rhythm of the heart.

2. The system of claim 1, wherein the instructions, when executed by the one or more processors, are further configured to receive reference signals from a reference electrode that is positioned within an inner volume of the expandable distal-end assembly such that reference electrode is a first distance from the first electrode and a second distance from the second electrode, the second distance being different than the first distance.

3. The system of claim 2, wherein a difference between the first distance and the second distance is less than a predefined threshold.

4. The system of claim 3, wherein the predefined threshold is less than 1 millimeter.

5. The system according to claim 2, wherein the reference electrode is disposed on a shaft, the expandable distal-end assembly being positioned at a distal end of the shaft.

6. The system according to claim 2, wherein the reference electrode is disposed approximately at a center of the inner volume of the expandable distal-end assembly.

7. The system according to claim 2, wherein the reference electrode is further configured as a reference electrode for applying a unipolar ablation signal between one or more ablation electrodes disposed on the expandable distal-end assembly and the reference electrode.

8. The system of claim 2, wherein at least one of the electrophysiological signals comprises a unipolar ECG signal, which is sensed in the tissue relative to the reference electrode.

9. The system according to claim 1, wherein the expandable distal-end assembly comprises at least first and second splines, and wherein the first and second electrodes are coupled to the first and second splines, respectively.

10. The system according to claim 2, wherein the expandable distal-end assembly is configured to transition between an expanded configuration and a collapsed configuration.

11. The system according to claim 10, wherein in the expanded configuration, the reference electrode is positioned at least 9 mm from the tissue.

12. The system according to claim 1, wherein the instructions, when executed by the one or more processors, are further configured to display at least the annotation in a bar graph.

13. The system according to claim 12, wherein the instructions, when executed by the one or more processors, are further configured to display in the bar graph at least a first annotation indicative of a first type of the timing error, and a second annotation which is indicative of a second type of the timing error that is different from the first type.

14. A medical system, comprising:
a catheter including:
a shaft extending along a longitudinal axis and configured for insertion into a heart of a patient;
a plurality of splines disposed near a distal end of the shaft and configured to bow radially outward when in an expanded configuration to define an inner volume;
a first electrode disposed on a first spline of the plurality of splines and configured to detect first electrophysiological signals;
a second electrode disposed on a second spline of the plurality of splines and configured to detect second electrophysiological signals;
a reference electrode disposed in the inner volume such that reference electrode is a first distance from the first electrode and a second distance from the second electrode, the second distance being different than the first distance;
one or more processors; and
a memory storing instructions that, when executed by the one or more processors are configured to cause the medical system to:
receive the first and the second electrophysiological signals;
calculate, based on the first and the second electrophysiological signals, one or more respective local activation times (LATs) which are indicative of an electrophysiological wave propagating in tissue;
identify, based at least in part on the calculated one or more respective LATs, a timing error of a rhythm of the heart; and
output for display an indication of the timing error identified in the rhythm of the heart.

15. The medical system of claim 14, wherein the instructions, when executed by the one or more processors, are further configured to cause the medical system to output a graph of at least one electrocardiogram (ECG) signal of the heart of the patient.

16. The medical system of claim 15, wherein the instructions, when executed by the one or more processors, are further configured to display the indication over the graph of the least one electrocardiogram (ECG) signal.

17. The medical system of claim 14, wherein the instructions, when executed by the one or more processors, are further configured to receive reference signals from the reference electrode.

18. The medical system of claim 17, wherein a difference between the first distance and the second distance is less than a predefined threshold.

19. The medical system according to claim 14, wherein the reference electrode is further configured as a reference electrode for applying a unipolar ablation signal between one or more ablation electrodes disposed on the plurality of splines and the reference electrode.

20. The medical system of claim 14, wherein at least one of the electrophysiological signals comprises a unipolar ECG signal, which is sensed in the tissue relative to the reference electrode.
